(19) United States
(12) Patent Application Publication
(10) Pub. No.: US 2021/0113543 A1
(43) Pub. Date: Apr. 22, 2021

(54) METHODS OF MONITORING, TREATING, AND PREVENTING RENAL INFLAMMATION FOLLOWING CARDIAC PROCEDURES OR EVENTS

(71) Applicant: Kantum Pharma, Inc., Concord, NH (US)

(72) Inventor: Sylvie Breton, Medford, MA (US)

(21) Appl. No.: 17/047,825

(22) PCT Filed: Apr. 16, 2019
(86) PCT No.: PCT/US2019/027649

§ 371 Date: Oct. 15, 2020

Related U.S. Application Data
(60) Provisional application No. 62/662,932, filed on Apr. 26, 2018.
(60) Provisional application No. 62/659,438, filed on Apr. 18, 2018.

(51) Int. Cl. A61K 31/451; G01N 33/66

(52) U.S. Cl. CPC A61K 31/451; G01N 2800/347; G01N 33/66

(57) ABSTRACT

The invention provides methods of monitoring development of renal inflammation in a subject who has undergone a cardiac procedure and has had a cardiac event by analyzing levels of one or more UDP-hexoses (such as UDP-glucose, UDP-galactose, UDP-glucuronic acid, N-acetyl-UDP-glucosamine and/or N-acetyl-UDP-galactosamine) in a sample from the subject. The invention also provides methods of treating or preventing renal inflammation in a subject who has had a cardiac procedure, will have a cardiac procedure, or has had a cardiac event by providing a P2Y 14 receptor antagonist to the subject. The invention further provides methods for evaluating cardiac health in a subject. Finally, the invention provides methods of monitoring one or more UDP-hexoses (such as UDP-glucose, UDP-galactose, UDP-glucuronic acid, N-acetyl-UDP-glucosamine and/or N-acetyl-UDP-galactosamine) in a subject.

### CROSS-REFERENCE TO RELATED APPLICATIONS

This application claims the benefit of, and priority to, U.S. Provisional Application No. 62/659,438, filed Apr. 18, 2018, and U.S. Provisional Application No. 62/662,932, filed Apr. 26, 2018, the contents of each of which are incorporated herein by reference.

### FIELD OF THE INVENTION

The invention generally relates to methods of monitoring, treating, and preventing renal inflammation following cardiac procedures or cardiac events.

### BACKGROUND

Acute kidney injury (AKI) afflicts up to 30% of patients following cardiac surgery and over 40% of patients following cardiac arrest. AKI impairs the function of other organs, including the brain, lungs, and gut. For heart surgery patients, AKI increases the risk of death during hospitalization by 500%, and patients who do recover from surgery are at increased risk of developing chronic kidney disease in the ensuing months.

Several factors make preventing AKI in cardiac patients difficult. First, many of the risk factors associated with AKI following a cardiac procedure or heart attack, such as age, hypertension, hyperlipidemia, peripheral vascular disease, and chronic renal disease, are not modifiable. In addition, other therapies designed to prevent AKI, such as high-dose perioperative atorvastatin and remote ischemic preconditioning, have shown little proven benefit.

Another reason that AKI is so problematic for cardiac patients is that existing methods do not allow for timely identification of AKI. Restoration of kidney function requires prompt detection of AKI, but an immediate diagnosis is not always possible. Elevated serum creatinine (SCr) concentration is a common diagnostic marker of AKI, but SCr is not a predictive biomarker and an increase in SCr indicative of AKI typically takes several days to develop, well after AKI has occurred. Reduced urine output, another symptom of AKI, can have other causes and therefore is not a conclusive indicator. Also, reduced urine output is also not predictive, and therefore AKI has already developed by the time reduced urine output is observed. Thus, barriers to prevention and diagnosis of AKI preclude effective treatment, and AKI continues to pose a serious health risk for individuals who have cardiac surgeries or heart attacks.

### SUMMARY

The invention provides methods that allow physicians to predict whether a cardiac patient is likely to develop AKI and to evaluate the cardiac health of a patient. The invention recognizes that renal inflammation contributes to the development and progression of AKI. The invention also recognizes that one or more UDP-hexoses (such as UDP-glucose, UDP-galactose, UDP-glucuronic acid, N-acetyl-UDP-glucosamine and/or N-acetyl-UDP-galactosamine) are agonists of the P2Y14 receptor and are predictive markers for development of renal inflammation and an indicator of cardiac health. Accordingly, by measuring levels of UDP-hexose, e.g., UDP-glucose, in patients who have had cardiac events or have undergone cardiac procedures, renal inflammation can be detected prior to the onset of AKI. Thus, the methods of the invention allow prognostication of, or detection of, AKI in time to provide therapeutic intervention to preserve and/or improve renal function. The methods also allow assessment of cardiac health.

Aspects of the invention are accomplished by monitoring patients who have undergone a cardiac procedure or have had a cardiac event for development of renal inflammation by analyzing UDP-hexose (such as UDP-glucose, UDP-galactose, UDP-glucuronic acid, N-acetyl-UDP-glucosamine and/or N-acetyl-UDP-galactosamine) levels. UDP-hexoses activate the purinergic receptor P2Y14 on renal tubular intercalated cells to promote renal inflammation, and patients who display elevated levels of urinary UDP-hexose, e.g., UDP-glucose, after a heart surgery or heart attack are at increased risk of developing renal inflammation, which may progress to AKI. The invention also provides methods of treating or preventing renal inflammation in cardiac patients by providing a P2Y14 antagonist after a surgical procedure or heart attack.

The methods of the invention may involve obtaining a sample from a subject who has undergone a cardiac procedure or has had a cardiac event, conducting an assay on the sample to measure the level of one or more UDP-hexoses (such as UDP-glucose, UDP-galactose, UDP-glucuronic acid, N-acetyl-UDP-glucosamine and/or N-acetyl-UDP-galactosamine) in the sample, and comparing the level of the one or more UDP-hexoses from the sample with a reference level of one or more UDP-hexoses. Based on the comparing step, the methods of the invention then allow for determining whether the subject is at risk of developing or has developed renal inflammation. For example, if the level of a UDP-hexose in the sample is elevated compared to a reference level, then the patient is at an increased risk of developing renal inflammation or is in the early stages of developing renal inflammation that may progress to acute kidney injury.

The subject may be a human of any age, e.g., a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The cardiac procedure may be an aneurysm repair, angioplasty, aorta transcatheter repair, biopsy, cardiomyoplasty, carotid endarterectomy, catheter repair, catheterization, catheterization, chemical cardioversion, congenital aortic stenosis surgery, congenital pulmonary stenosis balloon valvuloplasty, congenital pulmonary stenosis surgery, coronary artery bypass graft, electrical cardioversion, heart transplant, heart valve repair, heart valve replacement, implantation of a defibrillator, implantation of a stent, implantation of a stent, implantation of a total artificial heart, implantation of a ventricular assist device, implantation of pacemaker, pericardiectomy, pericardiocentesis, removal of implanted device, septal myectomy, surgery for pulmonary atresia, therapeutic hypothermia, or transmyocardial laser revascularization.

The cardiac event may be angina, myocardial infarction, stroke, heart failure, hemorrhagic stroke, hypertensive heart disease, rheumatic heart disease, cardiomyopathy, heart arrhythmia, congenital heart disease, valvular heart disease, carditis, aortic aneurysms, peripheral artery disease, subarachnoid hemorrhage, thromboembolic disease, thromboembolic stroke, traumatic brain injury, or venous thrombosis. In certain embodiments, the cardiac event is myocardial infarction.

The invention encompasses numerous methods for measuring and comparing UDP-hexose levels. In certain embodiments, a reference level is obtained or used. The reference level may be an average UDP-hexose level in a population of healthy subjects. The reference level may be a range of one standard deviation, two standard deviations, or three standard deviations from an average UDP-hexose level in a population of healthy subjects. The reference level may be the subject's own UDP-hexose level prior to undergoing the cardiac procedure. The reference level may be a range of one standard deviation, two standard deviations, or three standard deviations from the subject's own UDP-hexose level prior to undergoing the cardiac procedure. The reference level may be a cut-off concentration or range of concentrations below/above which the subject's risk of developing or having renal inflammation that may lead to AKI may be estimated or determined.

The methods of the invention can be conducted using various types of samples, so long as the sample includes a UDP-hexose (such as UDP-glucose, UDP-galactose, UDP-glucuronic acid, N-acetyl-UDP-glucosamine and/or N-acetyl-UDP-galactosamine). For example, the UDP-hexose may be UDP-glucose. UDP-glucose is present in many different body fluid samples. Exemplary body fluids that may contain UDP-glucose include urine, blood, plasma, serum, sweat, saliva, semen, feces, or phlegm. Preferably, the body fluid is blood or urine. UDP-glucose is also found in tissue. Accordingly, the sample may also be a tissue sample.

The methods may include monitoring the subject over time. In certain embodiments, monitoring may be accomplished by performing the method steps at multiple time points. For example, samples may be obtained at more than one time point, an assay may be conducted at each time point, and UDP-hexose levels may be compared to reference levels at each time point. UDP-hexose levels in sample taken from the subject at different time points may be compared to each other. The methods may include determining that the subject is at risk of developing or has developed renal inflammation if the levels of UDP-hexose in samples taken at different time points increase over time. The methods may include determining that renal inflammation may progress to AKI in the subject or determining the likelihood that that renal inflammation will progress to AKI in the subject.

The methods may include providing a P2Y14 antagonist to the subject if the subject has an elevated level of UDP-hexose (e.g. UDP-glucose). The P2Y14 antagonist may be a substituted 2-naphthoic acid. The P2Y14 antagonist may be 4-((piperidin-4-yl)-phenyl)-(7-(4-(trifluoromethyl)-phenyl)-2-naphthoic acid (PPTN).

In another aspect, the invention provides methods of treating or preventing renal inflammation in a subject who has undergone or will undergo a cardiac procedure or has experienced a cardiac event by providing a P2Y14 antagonist to the subject. The P2Y14 antagonist may be provided before, during, or after the cardiac procedure or at any combination of such time points.

The P2Y14 antagonist may be a substituted 2-naphthoic acid. The P2Y14 antagonist may be 4-((piperidin-4-yl)-phenyl)-(7-(4-(trifluoromethyl)-phenyl)-2-naphthoic acid (PPTN) or prodrug, analog, derivative, or pharmaceutically acceptable salt thereof.

The cardiac procedure or cardiac event may be any cardiac procedure or cardiac event described above. The cardiac procedure or cardiac event may be associated with elevated UDP-hexose levels in the subject. The renal inflammation may be associated with acute kidney injury. The methods may include measuring a level of UDP-hexose in a sample from a subject. The sample may be a body fluid sample, as described above. Preferably, the body fluid is blood or urine.

The P2Y14 antagonist may be provided is a formulation. The formulation may be an aqueous solution. The formulation may contain the P2Y14 antagonist at a certain concentration. For example, the P2Y14 antagonist may be present in the formulation at ≥0.001 μg/ml, ≥0.002 μg/ml, ≥0.005 μg/ml, ≥0.01 μg/ml, ≥0.02 μg/ml, ≥0.05 μg/ml, ≥0.1 μg/ml, ≥0.2 μg/ml, >0.5 μg/ml, ≥1 μg/ml, ≥2 μg/ml, ≥5 μg/ml, ≥10 μg/ml, ≥20 μg/ml, ≥50 μg/ml, ≥100 μg/ml, ≥200 μg/ml, ≥500 μg/ml, ≥1 mg/ml, ≥2 mg/ml, ≥5 mg/ml, or ≥10 mg/ml. The P2Y14 antagonist may be present in the formulation at from about 1 μg/ml to about 20 mg/ml, from about 2 μg/ml to about 20 mg/ml, from about 5 μg/ml to about 20 mg/ml, from about 10 μg/ml to about 20 mg/ml, from about 20 μg/ml to about 20 mg/ml, from about 50 μg/ml to about 20 mg/ml, from about 100 μg/ml to about 20 mg/ml, from about 200 μg/ml to about 20 mg/ml, from about 500 μg/ml to about 20 mg/ml, from about 1 mg/ml to about 20 mg/ml, from about 2 mg/ml to about 20 mg/ml, from about 5 mg/ml to about 20 mg/ml, from about 1 μg/ml to about 10 mg/ml, from about 2 μg/ml to about 10 mg/ml, from about 5 μg/ml to about 10 mg/ml, from about 10 μg/ml to about 10 mg/ml, from about 20 μg/ml to about 10 mg/ml, from about 50 μg/ml to about 10 mg/ml, from about 100 μg/ml to about 10 mg/ml, from about 200 μg/ml to about 10 mg/ml, from about 500 μg/ml to about 10 mg/ml, from about 1 mg/ml to about 10 mg/ml, from about 2 mg/ml to about 10 mg/ml, from about 5 mg/ml to about 10 mg/ml, from about 1 μg/ml to about 5 mg/ml, from about 2 μg/ml to about 5 mg/ml, from about 5 μg/ml to about 5 mg/ml, from about 10 μg/ml to about 5 mg/ml, from about 20 μg/ml to about 5 mg/ml, from about 50 μg/ml to about 5 mg/ml, from about 100 μg/ml to about 5 mg/ml, from about 200 μg/ml to about 5 mg/ml, from about 500 μg/ml to about 5 mg/ml, from about 1 mg/ml to about 5 mg/ml, from about 2 mg/ml to about 5 mg/ml, from about 1 μg/ml to about 2 mg/ml, from about 2 μg/ml to about 2 mg/ml, from about 5 μg/ml to about 2 mg/ml, from about 10 μg/ml to about 2 mg/ml, from about 20 μg/ml to about 2 mg/ml, from about 50 μg/ml to about 2 mg/ml, from about 100 μg/ml to about 2 mg/ml, from about 200 μg/ml to about 2 mg/ml, from about 500 μg/ml to about 2 mg/ml, or from about 1 mg/ml to about 2 mg/ml.

The formulation may contain an agent that increases the solubility of the P2Y14 antagonist in an aqueous solution. The agent may be α-tocopherol polyethylene glycol succinate (TPGS) or sulfobutyl ether beta-cyclodextrin (SBECD). The formulation may contain the agent at a certain concentration. For example, the agent may be present in the formulation at less than about 40%, less than about 35%, less than about 30%, less than about 25%, less than about 20%, less than about 15%, less than about 10%, less than about 5%, less than about 2%, less than about 1%, less than about 0.5%, less than about 0.2%, less than about 0.1%, less than about 0.05%, less than about 0.02%, less than about 0.01%, less than about 0.005%, less than about 0.002%, or less than about 0.001%. The agent may be present in the formulation at from about 0.001% to about 0.01%, from about 0.003% to about 0.03%, from about 0.01% to about 0.1%, from about 0.03% to about 0.03%, from about 0.1% to about 1%, from about 0.3% to about 3%, from about 1% to about 10%, from about 2% to about 10%, from about 3% to about 10%, from about 5% to about 10%, from about 5% to about 12%, from about 5% to about 15%, from about 5% to about 20%, from about 7.5% to about 10%, from about 7.5% to about 12%, from about 7.5% to about 15%, from about 7.5% to about 20%, from about 10% to about 12%, from about 10% to about 15%, or from about 10% to about 20%.

The formulation may have a pH in a physiologically-compatible range. The formulation may have a pH of ≥4.0, ≥4.5, ≥5.0, ≥5.5, ≥6.0, ≥6.5, ≥7.0, ≥7.5, or ≥8.0. The formulation may have a pH within a range. For example, the formulation may have a pH of from about 4.0 to about 9.0, from about 5.0 to about 9.0, from about 6.0 to about 9.0, from about 7.0 to about 9.0, from about 4.0 to about 8.0, from about 5.0 to about 8.0, from about 6.0 to about 8.0, from about 7.0 to about 8.0, from about 4.0 to about 7.0, from about 5.0 to about 7.0, or from about 6.0 to about 7.0. The formulation may have a pH of about 5.0, about 5.5, about 6.0, about 6.5, about 7.0, about 7.5, or about 8.0.

The formulation may contain a buffering agent and/or one or more salts. The buffering agent may be phosphate. The salt may be sodium chloride or potassium chloride. The formulation may contain saline or phosphate-buffered saline.

The formulation may contain dimethyl sulfoxide (DMSO). DMSO may be present in the formulation at less than about 10%, less than about 5%, less than about 3%, less than about 2%, less than about 1%, less than about 0.5%, less than about 0.3%, less than about 0.2%, or less than about 0.1%.

The formulation may be substantially free of solvents or other chemicals that may be toxic to a subject. For example, the formulation may be substantially free of dimethylacetamide (DMAc), ethanol, N-methylpyrrolidone (NMP), and/or polyethylene glycol (PEG).

In an aspect, the invention provides methods for monitoring for development of renal inflammation in a subject who has undergone a procedure that stresses the heart or cardiovascular system or has experienced an event that stresses the heart or cardiovascular system. The methods involve obtaining a sample from a subject who has undergone a procedure that stresses the heart or cardiovascular system or has experienced an event that stresses the heart or cardiovascular system, conducting an assay on the sample to measure the level of one or more UDP-hexose (such as UDP-glucose, UDP-galactose, UDP-glucuronic acid, N-acetyl-UDP-glucosamine and/or N-acetyl-UDP-galactosamine) in the sample, and comparing the level of the one or more UDP-hexoses from the sample with a reference level of one or more UDP-hexoses.

The procedure that stresses the heart or cardiovascular system may be thoracic surgery, abdominal surgery, or orthopedic surgery.

The event that stresses the heart or cardiovascular system may be lung inflammation or brain injury.

In an aspect, the invention provides methods for evaluating cardiac health in a subject. The methods include conducting an assay on a sample from the subject to measure a level of UDP-hexose in the sample from a subject and comparing the level of UDP-hexose in the sample with a reference level of UDP-hexose. An elevated level of UDP-hexose in the sample may indicate abnormal cardiac health in the subject. The methods may include repeating the conducting and comparing steps to monitor cardiac health in the subject over time.

The reference level of UDP-hexose level may be any level, such as those described above. The reference level may be an average UDP-hexose level in a population of healthy subjects. The reference level may be the subject's own UDP-hexose level in a sample obtained at an earlier time.

The sample may be any sample, such as those described above. For example, the sample may be a body fluid sample.

The subject may be known to have a cardiac condition. The subject may not be known to have a cardiac condition.

In an aspect, the invention provides methods of monitoring the level of a UDP-hexose in a subject. The methods include obtaining a sample from a subject comprising UDP-hexose, ionizing at least a portion of the sample to generate UDP-hexose ions, introducing the UDP-hexose ions into a mass spectrometer to obtain a level of UDP-hexose in the sample, and comparing the level of UDP-hexose in the sample to a reference level to monitor the level of UDP-hexose in the subject. The methods may include repeating the obtaining, ionizing, introducing, and comparing steps.

The reference level of UDP-hexose level may be any level, such as those described above. The reference level may be an average UDP-hexose level in a population of healthy subjects. The reference level may be the subject's own UDP-hexose level in a sample obtained at an earlier time.

The sample may be any sample, such as those described above. For example, the sample may be a body fluid sample. The body fluid may be blood, serum, urine, plasma, sweat, or saliva.

The methods may include introducing the sample into a liquid chromatography system. The sample may be introduced into the liquid chromatography system prior to the ionizing step.

### DETAILED DESCRIPTION

The invention provides methods of monitoring, treating, and preventing renal inflammation associated with cardiac events or cardiac procedures. Renal inflammation contributes to the development and progression of acute kidney injury (AKI), a common and serious secondary condition for cardiac patients. AKI occurs in up to 30% of patients who undergo cardiac procedures and up to 40% of patients who have suffered cardiac arrest. According to some reports, up to 5% of patients require renal replacement therapy, such as dialysis or kidney transplants, following heart surgery. In addition, patients who develop post-surgical AKI display increased rates of mortality, multiple organ failure, and chronic kidney disease.

Effective treatment of AKI requires immediate therapeutic intervention. This presents a conundrum for clinicians, however, because diagnostic markers known in the art, such as serum creatinine (SCr) concentration and urine output, require several hours or days to manifest AKI.

Recent reports have identified the purinergic receptor P2Y14, also called GPR105, as a key mediator of renal inflammation. The gene and protein for human P2Y14 are described in, for example, Entrez Gene ID no. 9934, GenBank ID no. D13626, RefSeq ID no. NM_014879, and UniProt ID no. NM_01487, the contents of which are incorporated herein by reference. P2Y14 is a G protein-coupled receptor expressed on the surface of intercalated cells (ICs) in the collecting duct system of the kidney. P2Y14 binds uridine diphosphate glucose (UDP-glucose), an ester of pyrophosphoric acid with the nucleoside uridine, as well as UDP-galactose, UDP-glucuronic acid, N-acetyl-UDP-glucosamine and/or N-acetyl-UDP-galactosamine. Abbracchio et al., Characterization of the UDP-glucose receptor (re-named here the P2Y14 receptor) adds diversity to the P2Y receptor family, Trends Pharmacol Sci. 2003 February; 24(2):52-5, DOI: 10.1016/S0165-6147(02)00038-X, the contents of which are incorporated herein by reference. UDP-glucose is released into extracellular fluids from damaged cells and in a regulated manner from intact cells. Binding of UDP-glucose to P2Y14 triggers ICs to produce chemokines that lead to infiltration of neutrophils into the renal medulla. See Azroyan et al., Renal Intercalated Cells Sense and Mediate Inflammation via the P2Y14 Receptor, PLoS ONE 10(3): e0121419 (2015), doi:10.1371/journal.pone.0121419. Thus, high levels of UDP-glucose activate P2Y14 to cause renal inflammation and contribute to AKI. In certain embodiments, the high UDP-glucose level is found in urine or pre-urine.

The invention addresses the difficulty of diagnosing and treating AKI in cardiac patients by measuring one or more UDP-hexoses (such as UDP-glucose, UDP-galactose, UDP-glucuronic acid, N-acetyl-UDP-glucosamine and/or N-acetyl-UDP-galactosamine) levels to monitor renal inflammation in such patients. The invention recognizes that measurement of one or more UDP-hexoses (such as UDP-glucose, UDP-galactose, UDP-glucuronic acid, N-acetyl-UDP-glucosamine and/or N-acetyl-UDP-galactosamine) levels in samples from patients who have had a cardiac procedure or a cardiac or cardiovascular event allows rapid detection of renal inflammation during the window of highest risk of AKI. Because renal inflammation can be detected before the patient has developed AKI, AKI can be treated more effectively. The invention further recognizes that providing a P2Y14 antagonist to such patients who display elevated levels of one or more UDP-hexoses (such as UDP-glucose, UDP-galactose, UDP-glucuronic acid, N-acetyl-UDP-glucosamine and/or N-acetyl-UDP-galactosamine) attenuates renal inflammation. Consequently, the invention provides methods that allow treatment of AKI in its incipient stages or prevention of AKI altogether.

Another insight of the invention is that one or more UDP-hexoses (such as UDP-glucose, UDP-galactose, UDP-glucuronic acid, N-acetyl-UDP-glucosamine and/or N-acetyl-UDP-galactosamine) are an indicator of cardiac health. In particular, elevated levels of one or more UDP-hexoses indicate poor or abnormal cardiac health. Thus, the invention therefore provides methods of evaluating cardiac health in a subject by analyzing levels of one or more UDP-hexoses in a sample obtained from the subject.

The invention also recognizes that providing a P2Y14 antagonist to a patient prior to surgery promotes the maintenance and restoration of circulation within organs, such as the brain, lung, and heart, during and after surgery, in addition to the kidney. Surgical procedures can inhibit the flow of blood to vital organs, and reperfusion of the organ can result in inflammation and oxidative damage due to oxidative stress. The invention therefore provides methods of administering a P2Y14 antagonist prophylactically to prevent or minimize reperfusion injury.

### Cardiac Procedures and Cardiac Events

The methods of the invention involve monitoring and treating subjects who have undergone a cardiac procedure, will undergo a cardiac procedure, or have experienced a cardiac or cardiovascular event.

The cardiac procedure may be a surgical or non-surgical procedure. Preferably, the cardiac procedure is surgical. For example and without limitation, the cardiac procedure may be a mitral valve replacement, aneurysm repair, angioplasty, aorta transcatheter repair, biopsy, cardiomyoplasty, carotid endarterectomy, catheter repair, catheterization, chemical cardioversion, congenital aortic stenosis surgery, congenital pulmonary stenosis balloon valvuloplasty, congenital pulmonary stenosis surgery, coronary artery bypass graft, electrical cardioversion, heart transplant, heart valve repair, heart valve replacement, implantation of a defibrillator, implantation of a stent, implantation of a stent, implantation of a total artificial heart, implantation of a ventricular assist device, implantation of pacemaker, pericardiectomy, pericardiocentesis, removal of implanted device, septal myectomy, surgery for pulmonary atresia, therapeutic hypothermia, or transmyocardial laser revascularization.

The methods may include other surgeries in which the heart or cardiovascular system is not the target organ system but which stress or otherwise affect the heart or cardiovascular system. For example and without limitation, the surgery may be thoracic surgery, abdominal surgery, or orthopedic surgery.

The cardiac event may be any severe or acute cardiovascular condition. For example and without limitation, the cardiac event may be angina, myocardial infarction, thromboembolic or hemorrhagic stroke, heart failure, hypertensive heart disease, rheumatic heart disease, cardiomyopathy, heart arrhythmia, congenital heart disease, valvular heart disease, carditis, aortic aneurysms, subarachnoid hemorrhage, peripheral artery disease, thromboembolic disease, traumatic brain injury, or venous thrombosis. Preferably, the cardiac event is myocardial infarction.

The methods may include events that primarily affect organ systems other than the cardiovascular system but stress the heart or cardiovascular system or have significant secondary effects on the heart or cardiovascular system. For example and without limitation, the event may include traumatic brain injury, lung inflammation, internal bleeding, or external bleeding.

### Measuring UDP-Hexose Levels

The level of one or more UDP-hexoses (such as UDP-glucose, UDP-galactose, UDP-glucuronic acid, N-acetyl-UDP-glucosamine and/or N-acetyl-UDP-galactosamine) may be measured by any suitable method. Examples below are discussed in the context of UDP-glucose. However, any method for measuring UDP-glucose is applicable to measuring any of the described UDP-hexoses. Preferably, UDP-glucose is measured by coupling a reaction converting UDP-glucose to a byproduct with the stoichiometric production of NADH or UDP as described in WO 2017/165665, the contents of which are incorporated herein in their entirety. UDP-glucose levels may also be measured using the protocols described in Barrett et al., Molec. Pharmacol., 2013, 84, 41-49, the contents of which are incorporated herein by reference in their entirety.

In certain embodiments that entail measurement of NADH as the readout molecule, the assay may include pre-processing steps remove proteins that interact with NADH production and/or endogenous NADH from the sample. High levels (e.g., >2 μM) of endogenous NADH from the sample can inhibit the assay. Alternatively or additionally, a control reaction lacking exogenous enzyme may be performed to measure the amount of pre-existing NADH in the sample, and this value can be subtracted from the value obtained from a reaction that receives exogenous enzyme. Next, the liquid sample is buffered to pH 8-9, for example, pH 8.0. The enzyme UDP-glucose dehydrogenase is added to the reaction along with the co-factor NAD⁺. During the reaction UDP-glucose is converted to UDP-glucuronic acid, and a stoichiometric amount of NAD is converted to NADH. NADH is then measured, and its concentration is used to deduce the starting UDP-glucose concentration. The amount of substrate and/or the reaction rate may be optimized so that the reaction occurs substantially in a substantially linear portion of the Michaelis-Menten graph.

In some embodiments, excess NAD is added to the reaction, along with enzyme in excess, such that UDP-glucose is limiting. For example, NAD can be added to a concentration of 2 mM per well, and 0.04 units of enzyme added per well to achieve an excess of both. One unit of enzyme is the amount of UDP-glucose dehydrogenase required to oxidize 1.0 mole of UDP-glucose to UDP-glucuronic acid per minute at pH 8.7 at 25° C.

Alternatively, the complete reaction curve can be determined for each sample and the data fit to a non-linear rate equation (e.g., “progress-curve analysis”). This is particularly useful when the slope of the linear region of the Michaelis-Menten kinetics curve for a desired enzyme is very steep (e.g., when the initial rate is too fast to measure accurately) or when an excess of substrate (e.g., NAM is used in the reaction mix

The methods may include lateral flow assays adapted for use in the detection of NADH or UDP. Such lateral flow assays permit the flow of a liquid sample, applied to the sample application zone, to deliver the sample/reactants to a test region (e.g., a reaction zone) of the lateral strip or device, and then the sample with a generated byproduct is delivered to a detection zone, which provides a readout (e.g., visual, optical, fluorescent, etc.). As one example, an assay may use reduction of nitro blue tetrazolium (NBT) by NADH to generate a colored product at a test region. As samples with generated NADH flow over a region with NBT (no color), the NBT is reduced to the blue form, which is visible on a strip

In some embodiments in which NBT is used to generate a detectable product, a reductase may be immobilized on the dipstick or test strip. The reductase may be a diaphorase, and it may be immobilized via adsorption or via immunocapture. As the NADH-containing solution flows through the region with the reductase enzyme, the NADH is oxidized and would reduce the NBT to the colored precipitate NBTH.

In some embodiments, the level of NADH or UDP in a sample is detected by a lateral flow assay test (LFA), or strip test. LFAs detect the presence or absence of an analyte, e.g. NADH or UDP, in a liquid sample. With a lateral flow method, a spatial separation is defined in the strips between the sample application zone and detection region. Most conventional lateral flow strips are designed for test samples that are readily available in large quantities (e.g., urine). Lateral flow immunoassays are described below, but lateral flow assays may also be adapted for the measurement of an analyte without the use of antibody. Both lateral flow immunoassays (e.g., using a UDP-glucose antibody) and lateral flow analyte assays (e.g., detection of NADH to measure UDP-glucose levels) are contemplated for use herein.

In LFAs the test sample flows along a solid substrate via capillary action. After the sample is applied to the lateral flow strip, it encounters a test region where an enzymatic reaction coupled to NADH or UDP production occurs and continues to a region comprising a detection reagent that permits detection of NADH or UDP. The liquid may go through one or more different regions on the lateral flow strip following the test region and prior to the detection region.

LFAs are adapted to operate along a single axis to suit the test strip format or a dipstick format. Typically, LFAs proceed from sample application to readout without additional steps by the user, so sample application generally leads to an assay result with the further user input. Other lateral flow configurations may include one or more steps by the user after sample application, e.g., insertion into a detector device (e.g., a luminometer, fluorescence detector, etc.) or addition of another reagent. Strip tests are extremely versatile and can be easily modified by one skilled in the art for detecting an enormous range of antigens or analytes from fluid samples such as urine, blood, water samples etc. Strip tests are also known as “dipstick tests,” the name bearing from the literal action of “dipping” the test strip into a fluid sample to be tested. LFA strip tests are easy to use, require minimum training and can easily be included as components of point-of-care test (POCT) diagnostics to be used on site in the field.

A typical test strip may comprise one or more of following components: (1) sample application zone comprising e.g., an absorbent pad (i.e., the matrix or material) onto which the test sample is applied; (2) test region comprising immobilized enzyme; (3) a test results area comprising a detection reagent or reaction membrane—such as a hydrophobic nitrocellulose or cellulose acetate membrane onto which, for example, a detection reagent is immobilized in a line across the membrane as a capture zone or test line (a control zone may also be present, containing NADH or another reducing agent, for example, that reduces NBT to generate a blue color) or an antibody reagent; and (4) optional wick or waste reservoir—a further absorbent pad designed to draw the sample across the detection reagent zone or reaction membrane by capillary action and collect it. In addition, lateral flow strips as described herein may further comprise one or more of the following: a region comprising a strong base or a region comprising immobilized NAD nucleosidase to degrade unreacted NAD⁺.

The components of the strip may be fixed to an inert backing material and may be presented in a simple dipstick format or within a plastic casing with a sample port and reaction window showing the test readout/capture and control zones. The test may incorporate a second, coated line which contains an antibody or other reagent that picks up free readout substrate (e.g., free latex or gold particles) in order to confirm the test has operated correctly.

The use of “dip sticks” or LFA test strips and other solid supports has been described in the art in the context of an immunoassay for a number of antigen biomarkers. U.S. Pat. Nos. 4,943,522; 6,485,982; 6,187,598; 5,770,460; 5,622,871; and 6,565,808, and U.S. patent application Ser. Nos. 10/278,676; 09/579,673; and Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to, U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. Given the reaction description and considerations described herein, it is within the skill of one in the art to modify the teachings regarding “dip stick” technology for the detection of NADH or UDP using e.g., dye, luciferin or fluorescent reagents as described herein.

In some embodiments, the reaction to generate a stoichiometric amount of NADH from the reaction of UDP-glucose with UDP-glucose dehydrogenase is incubated for a matter of minutes, e.g., 5 or 10 minutes, in the liquid assay format in order to generate sufficient amounts of NADH for detection. This extended time is not as readily achieved in the dipstick or lateral flow format. However, options to overcome this include performing the first enzymatic reaction in an assay well for a prescribed period of time before inserting a dipstick or applying sample to a test strip. Alternatively, if all reactions took place on the dipstick or test strip, a shorter incubation should not present a problem because most of the enzyme reaction actually takes place within the first minute, although the reaction continues to remain linear after a 5-minute incubation, after the initial linear velocity for low (physiological) concentrations of UDP-glucose (up to 100 μM).

A urine dipstick is a colorimetric chemical assay comprising a reagent stick-pad. The dipstick is typically immersed in a fresh urine specimen and then withdrawn. Alternatively, the urine sample may be applied directly to the sample application zone by the subject (e.g., analogous to a pregnancy test). After predetermined times the colors of the reagent pad are compared to standardized reference charts. The urine dipstick offers an inexpensive and fast method to perform screening urinalyses, which helps in identifying the presence of various diseases or health problems. A urine dipstick provides a simple and clear diagnostic guideline and may be used in the methods and kits as described herein. Accordingly, one aspect of the present technology relates to a method for detecting NADH or UDP using a device, such as a dipstick, as described herein. When the sample is not clear, a centrifugation or filtration step to render a clear sample may be applied so as to avoid pigment or other entities from fouling the optical readout.

In some cases, the lateral flow strip may also comprise a control that gives a signal to the user that the assay is performing properly. For instance, the control zone may contain an immobilized receptive material that is generally capable of forming a chemical and/or physical bond with probes or with the receptive material immobilized on the probes. Some examples of such receptive materials include, but are not limited to, antigens, haptens, antibodies, protein A or G, avidin, streptavidin, secondary antibodies, and complexes thereof. In addition, it may also be desired to utilize various non-biological materials for the control zone receptive material. For instance, in some embodiments, the control zone receptive material may also include a polyelectrolyte that may bind to uncaptured probes. Because the receptive material at the control zone is only specific for probes, a signal forms regardless of whether the analyte is present. The control zone may be positioned at any location along the test strip, but is preferably positioned downstream from the detection zone.

In some embodiments, detection involves reduction of nitro blue tetrazolium by NADH present and/or generated during the assay. In such embodiments, the control line may include a line of NBT spatially downstream of the test line and immediately downstream of a line or zone of dried reducing agent. Flow of sample past the test line will liberate the reducing agent and carry it to the control line of NBT, which will be reduced to generate a control line indicating the sample reactants have successfully reacted at that point.

Qualitative, semi-quantitative, and quantitative results may be obtained with the lateral flow assays described herein. For example, when it is desired to semi-quantitatively or quantitatively detect an analyte, the intensity of any signals produced at the region comprising a detection reagent may be measured with e.g., an optical reader. The actual configuration and structure of the optical reader may generally vary as is readily understood by those skilled in the art. For example, optical detection techniques that may be utilized include, but are not limited to, luminescence (e.g., fluorescence, phosphorescence, etc.), absorbance (e.g., fluorescent or non-fluorescent), diffraction, etc. Further optical methods include but are not limited to, measurement of light scattering or simple reflectance, e.g., using a luminometer or photomultiplier tube; radioactivity, e.g., using a Geiger counter; electrical conductivity or dielectric capacitance; and release of electroactive agents, such as indium, bismuth, gallium or tellurium ions.

Once the amount of detection agent has been quantified, the amount may then be mapped onto another measurement scale. For example, while the result of the assay may be measured as a density of reflectance (Dr), the result reported may be more meaningful in other units, such as RI (intensity relative to that of a control zone or background level). Results may also be expressed as the number of copies of analyte present in the measurement volume.

The methods may include lateral flow immunoassays (LFIAs), in which antibodies that bind a target analyte are used in a competitive or sandwich immunoassay adapted to the lateral flow format. Conventional sandwich LFIAs are similar to sandwich ELISAs. The sample first encounters and mobilizes colored particles which are labeled with antibodies raised to the target antigen. The test line will also contain antibodies to the same target, although it may bind to a different epitope on the antigen. The test line will show as a colored band in positive samples, resulting from the accumulation or capture of antibody-bearing colored particles. In some embodiments, the lateral flow immunoassay may be a double antibody sandwich assay, a competitive assay, a quantitative assay or variations thereof. Conventional competitive LFIAs are similar to competitive ELISA. The sample first encounters colored particles which are labeled with the target antigen or an analogue. The test line contains antibodies to the target/its analogue. Unlabeled antigen in the sample will block the binding sites on the antibodies preventing capture of the colored particles at the test line. The test line will show as a colored band in negative samples. There are a number of variations on lateral flow technology. It is also possible to apply multiple capture zones to create a multiplex test.

Any substance generally capable of producing a signal that is detectable visually or by an instrumental device may be used as a detection reagent. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances are described in U.S. Pat. Nos. 5,670,381 and 5,252,459, which are incorporated herein in their entirety by reference. If the detectable substance is colored, the ideal electromagnetic radiation is light of a complementary wavelength. For instance, blue detection probes strongly absorb red light.

In some embodiments, the detectable substance may be a luminescent compound that produces an optically detectable signal. For example, suitable fluorescent molecules may include, but are not limited to, fluorescein, europium chelates, phycobiliprotein, rhodamine, and their derivatives and analogs. Other suitable fluorescent compounds are semiconductor nanocrystals commonly referred to as “quantum dots.”

In another embodiment, the detection agent is a particle. Examples of particles useful in the methods, assays and kits described herein include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads.

Further, suitable phosphorescent compounds include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or non-aqueous environment. For example, some suitable examples of ligands include, but are not limited to, pyridine; pyrazine; isonicotinamide; imidazole; bipyridine; terpyridine; phenanthroline; dipyridophenazine; porphyrin, porphine, and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and II, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. Nos. 4,614,723; 5,464,741; 5,518,883; 5,922,537; 6,004,530; and 6,582,930, which are incorporated herein in their entirety by reference.

Bipyridine metal complexes may also be utilized as phosphorescent compounds. Some examples of suitable bipyridine complexes include, but are not limited to, bis[(4,4′-carbomethoxy)-2,2′-bipyridine]2-[3-(4-methyl-2,2′-bipyridine-4-yl)propyl]-1,3-dioxolane ruthenium (II); bis(2,2′bipyridine)[4-(butan-1-al)-4′-methyl-2,2′-bi-pyridine]ruthenium (II); bis(2,2′-bipyridine)[4-(4′-methyl-2,2′-bipyridine-4′-yl)-butyric acid] ruthenium (II); tris(2,2′bipyridine)ruthenium (II); (2,2′-bipyridine)[bis-bis(1,2-diphenylphosphino)ethylene]2-[3-(4-methyl-2,2′-bipyridine-4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4(4′-methyl-2,2′-bipyridine-4-yl)butane]ruthenium (II); bis(2,2′-bipyridine)maleimidohexanoic acid, 4-methyl-2,2′-bipyridine-4′-butylamide ruthenium (II), and so forth.

An immunoassay measures the concentration of a substance in a sample, typically a fluid sample, using the interaction of an antibody or antibodies to its antigen. The assay takes advantage of the highly specific binding of an antibody with its antigen. In some embodiments, specific binding of a UDP molecule with an anti-UDP antibody forms a UDP-antibody complex. The complex may then be detected by a variety of methods known in the art. An immunoassay also often involves the use of a detection antibody. Antibodies contemplated for use with the methods and assays described herein include an anti-UDP-glucose antibody, an anti-UDP antibody, and anti-UDP-glucuronic acid antibody. Such antibodies may be designed and generated using methods known in the art and/or described herein.

In one embodiment, the antibody is detectably labeled or capable of generating a detectable signal. In one embodiment, the antibody is fluorescently labeled.

In some embodiments, levels of a desired biomarker or analyte (e.g., UDP-glucose, UDP, etc.) are measured by ELISA, also called enzyme immunoassay or EIA. ELISA is a biochemical technique that detects the presence of an antibody or an antigen in a sample.

In one embodiment, an ELISA involving at least one antibody with specificity for the particular desired antigen may be performed. A known amount of sample and/or antigen is immobilized on a solid support (e.g., a polystyrene micro titer plate). Immobilization may be either non-specific (e.g., by adsorption to the surface) or specific (e.g., where another antibody immobilized on the surface is used to capture antigen or a primary antibody). After the antigen is immobilized, the detection antibody is added, forming a complex with the antigen. The detection antibody may be covalently linked to an enzyme, or may itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample.

Older ELISAs utilize chromogenic substrates, though newer assays employ fluorogenic substrates with much higher sensitivity.

In one embodiment, a sandwich ELISA is used, where two antibodies specific for the target may be used. There are other different forms of ELISA, which are well known to those skilled in the art. Standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22: 895-904.

Antibodies or portions thereof may be used in immunoassays. For example, the immunoassay may use a complete immunoglobulin, antigen-binding fragment (Fab), Fab₂, variable domain (Fv), single chain variable fragment (scFv), third-generation (3G) antibody. The antibodies may be natural monoclonal antibodies or synthetic antibodies, such as recombinant antibodies, non-immunoglobulin derived synthetic antibodies, or affimer proteins. Methods of making monoclonal antibodies are known in the art and described in, for example, Antibodies: A Laboratory Manual, Second edition, edited by Greenfield, Cold Spring Harbor Laboratory Press (2014) ISBN 978-1-936113-81-1. Methods of making synthetic antibodies are described in, for example, US 2014/0221253; US 2016/0237142; and Miersch and Sidhu, Synthetic antibodies: concepts, potential and practical considerations, Methods. 2012 August; 57(4):486-98. doi: 10.1016/j.ymeth.2012.06.012, the contents of each of which are incorporated herein by reference.

In some embodiments, UDP-glucose or another molecule that serves as an indicator of UDP-glucose is detected by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi:10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi:10.1002/jms.1383, the contents of which are incorporated herein by reference.

The assays described herein may be adapted to be performed on an automated device platform that is programmed to automatically add, transfer and optionally, mix liquid samples or reaction mixtures, for example, in wells of a multiwell plate. The wells may include reagents as necessary, either added in liquid/solution form or, for example, dried or immobilized on a surface within the wells. Automated platforms that include liquid handling modules as well as detection (e.g., fluorescence, luminescence, absorbance, reflectance, etc.) modules are known to those of skill in the art. As but one non-limiting example, one might use, e.g., a Beckman Coulter AU5800 device. When adapted to an automated design, multiwell plates may include, in addition to test wells for assaying an unknown test sample, control wells including, e.g., blanks lacking enzyme or other reagents, to permit, among other things, the determination of background levels of, e.g., intermediate or surrogate indicator NADH. Other controls may include, e.g., positive control wells including a known amount of UDP-glucose; a set of separate positive control wells may include varying known amounts of UDP-glucose to establish a standard curve, e.g., over one or a plurality of orders of magnitude, that is read by the device and used to calculate amounts of UDP-glucose in the unknown test samples.

### Samples

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. The sample may also be a solid or semi-solid sample, such as a tissue sample, feces sample, or stool sample, that has been treated to take a liquid form by, for example, homogenization, sonication, pipette trituration, cell lysis etc. For the methods described herein, it is preferred that a sample is from urine, serum, whole blood, or sputum.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal (such as a mammal, such as a human). The subject may be a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after a cardiac procedure. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more before a cardiac procedure, or it may be obtained 1 hour, 2 hours, 4 hours, 6 hours, 8 hours, 12 hours, 24 hours, 36 hours, 2 days, 3 days, 4 days, 5 days, 6 days, 7 days or more after a cardiac procedure.

The sample may be obtained from an individual after a cardiac or vascular event. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more after a cardiac or vascular event.

### Reference Levels

The reference level may be defined based on clinical trials that determine the concentration of one or more UDP-hexoses (such as UDP-glucose, UDP-galactose, UDP-glucuronic acid, N-acetyl-UDP-glucosamine and/or N-acetyl-UDP-galactosamine) that optimally defines a cut-off point above which the likelihood of occurrence of AKI is high (the sensitivity) and below which the likelihood of occurrence of AKI is low (the specificity).

The reference level of UDP-hexose may be defined by a statistic describing the distribution of levels in normal healthy subjects. For example, the reference level may be an average level of UDP-hexose in a sample from a normal healthy subject or a population of normal healthy subjects. The reference level of UDP-hexose may be an average level of UDP-hexose in a sample from a subject who has not undergone a cardiac procedure or a population of subjects who have not undergone a cardiac procedure. The reference level of UDP-hexose may be an average level of UDP-hexose in a sample from a subject who has not had a cardiac event or a population of subjects who have not had a cardiac event. The reference level of UDP-hexose may be an average level of UDP-hexose in a sample from one or more subjects who have undergone a cardiac or other procedure but did not develop AKI. The reference level of UDP-hexose may be an average level of UDP-hexose in a sample from one or more subjects who have had a cardiac or other event but did not develop AKI.

The reference level may be above the highest observed level of UDP-hexose in a sample from a normal healthy subject or a population of normal healthy subjects. Any level above the reference level may be deemed to be significantly different from the average level of UDP-hexose in a sample from a normal healthy subject or a population of normal healthy subjects. The reference level may be greater than 95% of the levels observed in samples from a normal healthy subject or a population of normal healthy subjects, or it may be above the lower limit of the highest decile, quartile or tertile of the levels observed in samples from a normal healthy subject or a population of normal healthy subjects.

The reference level may be at least one standard deviation, at least two standard deviations, or at least three standard deviations above the average level of UDP-hexose in a sample from a normal healthy subject or a population of normal healthy subjects. Any level above the reference level may be deemed to be significantly different from the average level of UDP-hexose in a sample from a normal healthy subject or a population of normal healthy subjects.

The reference level may be at least one standard deviation, at least two standard deviations, or at least three standard deviations above the average level of UDP-hexose in a sample from a subject that has not undergone a cardiac procedure or a population of subjects that have not undergone a cardiac procedure. Any level above the reference level may be deemed to be significantly different from the average level of UDP-hexose from a subject who has not undergone a cardiac procedure or a population of subjects who have not undergone a cardiac procedure.

The reference level may be at least one standard deviation, at least two standard deviations, or at least three standard deviations above the average level of UDP-hexose in a sample from one or more subjects who have undergone a cardiac or other procedure but did not develop AKI. Any level above the reference level may be deemed to be significantly different from the average level of UDP-hexose from one or more subjects who have undergone a cardiac or other procedure but did not develop AKI.

The reference level may be at least one standard deviation, at least two standard deviations, or at least three standard deviations above the average level of UDP-hexose in a sample from a subject who has not had a cardiac event or a population of subjects who have not had a cardiac event. Any level above the reference level may be deemed to be significantly different from the average level of UDP-hexose in a subject who has not had a cardiac event or a population of subjects who have not had a cardiac event.

The reference level may be at least one standard deviation, at least two standard deviations, or at least three standard deviations above the average level of UDP-hexose in a sample from one or more subjects who have had a cardiac or other event but did not develop AKI. Any level above the reference level may be deemed to be significantly different from the average level of UDP-hexose from one or more subjects who have had a cardiac or other event but did not develop AKI.

The reference level may be a level of UDP-hexose in a control sample, a pooled sample of control individuals, or a numeric value or range of values based on the same. It is also contemplated that a set of standards may be established with reference levels providing thresholds indicative of the severity of renal inflammation.

The reference level may be a level of UDP-hexose in a sample of the same subject measured at an earlier time point. The reference level may be a level of UDP-hexose in a sample obtained from the same subject before or during a cardiac procedure. The reference level may be a level of UDP-hexose in a sample obtained from the same subject before or during a cardiac event. The reference level may be from a sample obtained 1 hour, 2 hours, 4 hours, 6 hours, 8 hours, 12 hours, 24 hours, 36 hours, 2 days, 3 days, 4 days, 5 days, 6 days, 7 days or more before a cardiac procedure.

The reference level may be at least one standard deviation, at least two standard deviations, or at least three standard deviations above a level of UDP-hexose in a sample obtained from the same subject at an earlier time point. Any level above the reference level may be deemed to be significantly different from the level in the earlier sample.

In some embodiments, the level of UDP-hexose measured in a sample from a subject identified as having renal inflammation may be at least 5%, at least 10%, at least 20%, at least 30%, at least 40%, at least 50%, at least 60%, at least 70%, at least 80%, at least 90%, at least 100%, at least 150%, at least 200%, or at least 300% higher than the reference level.

In some embodiments, the level of UDP-hexose measured in a sample from a subject identified as having abnormal cardiac health may be at least 5%, at least 10%, at least 20%, at least 30%, at least 40%, at least 50%, at least 60%, at least 70%, at least 80%, at least 90%, at least 100%, at least 150%, at least 200%, or at least 300% higher than the reference level.

The reference level may be adjusted to account for variables such as sample type, gender, age, weight, and ethnicity. Thus, reference levels accounting for these and other variables may provide added accuracy for the methods described herein.

The methods may include making a diagnosis, prediction, or prognostication regarding the subject based on a comparison of a measured level of UDP-hexose to a reference level. The prediction or prognostication may include a probability, e.g., a statistical value. The diagnosis, prediction, or prognostication may indicate the presence of, or likelihood of developing, a condition. The condition may be renal inflammation, acute kidney injury, or a stage or category of renal inflammation or acute kidney injury.

### Computer Systems for Measuring UDP-Hexose Levels

In some embodiments of the assays and/or methods described herein, the assay/method comprises or consists essentially of a system for determining (e.g. transforming and measuring) the level of UDP-hexose as described herein and comparing it to a reference level. If the comparison system, which may be a computer implemented system, indicates that the amount of the measured level of UDP-hexose is statistically higher than that of the reference amount, the subject from which the sample is collected may be identified as having renal inflammation.

In one embodiment, provided herein is a system comprising: (a) at least one memory containing at least one computer program adapted to control the operation of the computer system to implement a method that includes (i) a determination module configured to measure the level of UDP-hexose in a test sample obtained from a subject; (ii) a storage module configured to store output data from the determination module; (iii) a computing module adapted to identify from the output data whether the measured level of UDP-hexose in the test sample obtained from the subject is higher, by a statistically significant amount, than a reference level, and to provide a retrieved content; (iv) a display module for displaying for retrieved content (e.g., the amount of the measured level of UDP-hexose, or whether the measured level of UDP-hexose is higher than the reference level); and (b) at least one processor for executing the computer program.

Embodiments may be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions may be carried out by the execution of various code portions stored on various media and executed at various times. Furthermore, it should be appreciated that the modules may perform other functions, thus the modules are not limited to having any particular functions or set of functions.

The computer-readable storage media may be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon may be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments may include at minimum a determination module, a storage module, a computing module, and a display module. The functional modules may be executed on one, or multiple, computers, or by using one, or multiple, computer networks. The determination module has computer executable instructions to provide e.g., levels of expression products etc in computer readable form.

The determination module may comprise any system for detecting a signal resulting from the detection of UDP-hexose in a biological sample. In some embodiments, such systems may include an instrument, e.g., a plate reader for measuring absorbance. In some embodiments, such systems may include an instrument, e.g., the Cell Biosciences NANOPRO 1000™ System (Protein Simple; Santa Clara, Calif.) for quantitative measurement of proteins.

The information determined in the determination system may be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, patient name, and numerical value of the level of UDP-hexose. Such information may be provided in digital form that may be transmitted and read electronically, e.g., via the Internet, on diskette, via USB (universal serial bus) or via any other suitable mode of communication. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as levels of UDP-hexose in healthy subjects. In some embodiments, the storage module stores the information such as levels of UDP-hexose measured from the same subject in earlier time points.

The computing module may use a variety of available software programs and formats for computing the levels of UDP-hexose. Such algorithms are well established in the art. A skilled artisan is readily able to determine the appropriate algorithms based on the size and quality of the sample and type of data. The data analysis may be implemented in the computing module. In one embodiment, the computing module further comprises a comparison module, which compares the level of UDP-hexose in the test sample obtained from a subject as described herein with the reference level. For example, when the level of UDP-hexose in the test sample obtained from a subject is measured, a comparison module may compare or match the output data, e.g. with the reference level. In certain embodiments, the reference level has been pre-stored in the storage module. During the comparison or matching process, the comparison module may determine whether the level of UDP-hexose in the test sample obtained from a subject is higher than the reference level to a statistically significant degree. In various embodiments, the comparison module may be configured using existing commercially-available or freely-available software for comparison purpose, and may be optimized for particular data comparisons that are conducted.

The computing and/or comparison module, or any other module, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment, users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module may be the relative levels of UDP-hexose in the test sample obtained from a subject as compared to a reference level. In certain embodiments, the content displayed on the display module may indicate whether the levels of UDP-hexose are found to be statistically significantly higher in the test sample obtained from a subject as compared to a reference level. In some embodiments, the content displayed on the display module may show the levels of UDP-hexose from the subject measured at multiple time points, e.g., in the form of a graph. In some embodiments, the content displayed on the display module may indicate whether the subject has renal inflammation. In certain embodiments, the content displayed on the display module may indicate whether the subject is in need of a treatment for renal inflammation.

In one embodiment, the content based on the computing and/or comparison result is displayed on a computer monitor. In one embodiment, the content based on the computing and/or comparison result is displayed through printable media. The display module may be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules may be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the technology relating to determining the levels of UDP-hexose, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Methods of Providing a P2Y14 Receptor Antagonist

The invention provides methods of treating or preventing renal inflammation to a cardiac patient by providing a purinergic P2Y14 receptor antagonist, referred to simply as a P2Y14 antagonist. For subjects undergoing a cardiac procedure, the P2Y14 antagonist may be provided before, during, or after the cardiac procedure. For example and without limitation, the P2Y14 antagonist may be provided 1 hour, 2 hours, 4 hours, 6 hours, 8 hours, 12 hours, 24 hours, 36 hours, 2 days, 3 days, 4 days, 5 days, 6 days, 7 days or more before a cardiac procedure, or it may be provided 1 hour, 2 hours, 4 hours, 6 hours, 8 hours, 12 hours, 24 hours, 36 hours, 2 days, 3 days, 4 days, 5 days, 6 days, 7 days or more after a cardiac procedure

For subjects who have experienced a cardiac event, the P2Y14 antagonist may be provided at some interval in relation to the cardiac event. Alternatively or additionally, the P2Y14 antagonist may be provided at some interval relative to another preceding event, such as admission to a hospital, clinic, medical facility, or any unit thereof. The interval may be 1 hour, 2 hours, 4 hours, 6 hours, 8 hours, 12 hours, 24 hours, 36 hours, 48 hours, or 72 hours.

Providing the P2Y14 antagonist to the subject may include administering it to the subject. The P2Y14 antagonist may be administered by any suitable means. For example and without limitation, the P2Y14 antagonist may be administered orally, intravenously, enterally, parenterally, dermally, buccally, topically, transdermally, by injection, intravenously, subcutaneously, nasally, pulmonarily, or with or on an implantable medical device (e.g., stent or drug-eluting stent or balloon equivalents).

The P2Y14 antagonist may be provided at any suitable dosage. For example and without limitation, the P2Y14 antagonist may be provided at from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, or from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, or from 4.5 g/kg body weight to 5 g/kg body weight. These doses may be administered at a single time or multiple times each day, or may be administered continuously, for example by continuous intravenous infusion. For example and without limitation, the P2Y14 antagonist may be provided at from 0.001 mg/kg body weight/day (mg/kg/day) to 5 g/kg body weight/day. In some embodiments, the dosage range is from 0.001 mg/kg body weight/day to 1 g/kg body weight/day, from 0.001 mg/kg body weight/day to 0.5 g/kg body weight/day, from 0.001 mg/kg body weight/day to 0.1 g/kg body weight/day, from 0.001 mg/kg body weight/day to 50 mg/kg body weight/day, from 0.001 mg/kg body weight/day to 25 mg/kg body weight/day, from 0.001 mg/kg body weight/day to 10 mg/kg body weight/day, from 0.001 mg/kg body weight/day to 5 mg/kg body weight/day, from 0.001 mg/kg body weight/day to 1 mg/kg body weight/day, from 0.001 mg/kg body weight/day to 0.1 mg/kg body weight/day, or from 0.001 mg/kg body weight/day to 0.005 mg/kg body weight/day. Alternatively, in some embodiments the dosage range is from 0.1 g/kg body weight/day to 5 g/kg body weight/day, from 0.5 g/kg body weight/day to 5 g/kg body weight/day, from 1 g/kg body weight/day to 5 g/kg body weight/day, from 1.5 g/kg body weight/day to 5 g/kg body weight/day, from 2 g/kg body weight/day to 5 g/kg body weight/day, from 2.5 g/kg body weight/day to 5 g/kg body weight/day, from 3 g/kg body weight/day to 5 g/kg body weight/day, from 3.5 g/kg body weight/day to 5 g/kg body weight/day, from 4 g/kg body weight/day to 5 g/kg body weight/day, or from 4.5 g/kg body weight/day to 5 g/kg body weight/day. Effective doses may be estimated from dose-response relationships derived from in vitro or animal model test bioassays or systems or from clinical trials of the P2Y14 antagonist. The dosage should not be so large as to cause unacceptable adverse side effects.

### P2Y14 Antagonists

The P2Y14 antagonist may be any entity that interferes with ligand-binding, activation, or signaling by P2Y14. The P2Y14 antagonist may be a small or large organic or inorganic molecule. Preferably, the P2Y14 antagonist is a 4,7-disubstituted naphthoic acid derivative, such as one of the compounds described in U.S. Publication No. 2010/0298347, the contents of which are incorporated herein by reference. Such compounds may be represented by formula (I):

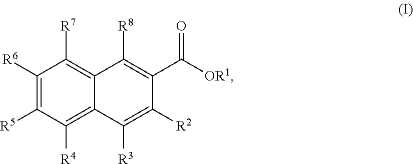

wherein:

R¹ is selected from the group consisting of hydrogen, C₃₋₆ cycloalkyl, benzyl, and C₁₋₆ alkyl wherein alkyl is optionally substituted with hydroxy, amino, C₁₋₄ alkylamino, di-(C₁₋₄ alkyl)amino, aminocarbonyl, C₁₋₄ alkylaminocarbonyl, di-(C₁₋₄alkyl)aminocarbonyl, C₁₋₄ alkylcarbonyloxy, C₁₋₄ alkyloxy, or one to five fluorines;

R² is hydrogen, fluorine, or hydroxy;

R³ is selected from the group consisting of: —(CH₂)ₘaryl, —(CH₂)ₘheteroaryl, —OCH₂-aryl, —OCH₂-heteroaryl, —(S)ᵣCH₂-aryl, —(S),CH₂-heteroaryl, —CH₂O—aryl, —CH₂O—heteroaryl, —CH₂(S)ᵣaryl, and —CH₂(S)ᵣ-heteroaryl;

wherein any methylene (CH₂) carbon atom in R3 is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group; and wherein aryl and heteroaryl are optionally substituted with one to three R^{c} substituents independently selected from the group consisting of:

halogen,

cyano,

nitro,

C₁₋₆ alkoxy, wherein alkoxy is optionally substituted with one to five substituents independently selected from fluorine, hydroxy, and C₁₋₃ alkoxy,

C₁₋₆ alkyl, wherein alkyl is optionally substituted with one to five substituents independently selected from fluorine, hydroxy, and C₁₋₃ alkoxy,

C₂₋₆ alkenyl, wherein alkenyl is optionally substituted with one to five substituents independently selected from fluorine, hydroxy, and C₁₋₃ alkoxy,

(CH₂)ₙ-aryl,

(CH₂)ₙ-heteroaryl,

(CH₂)ₙ-heterocyclyl,

(CH₂)ₙ—C₃₋₆ cycloalkyl,

(CH₂)ₙ—OR⁹,

(CH₂)ₙ—CO₂R⁹,

(CH₂)ₙ—N(R⁹)₂,

(CH₂)ₙ—CON(R⁹)₂,

(CH₂)ₙ—OCON(R⁹)₂,

(CH₂)ₙ—SO₂N(R⁹)₂,

(CH₂)ₙ—SO₂N(R⁹)C(O)R⁹,

(CH₂)ₙ—C(O)ₙ(R⁹)SO₂R¹⁰,

(CH₂)ₙ—S(O)ᵣR¹⁰,

(CH₂)ₙ—NR¹¹SO₂R¹⁰;

(CH₂)ₙ—NR¹¹CON(R⁹)₂,

(CH₂)ₙ—NR¹¹COR⁹, and

(CH₂)ₙ—NR¹¹CO₂R¹⁰;

wherein aryl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R^{c} is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group;

R⁴, R⁵, R⁷, and R⁸ are each independently selected from the group consisting of:

hydrogen,

halogen,

C₁₋₄ alkyl, optionally substituted with one to five fluorines,

C₁₋₄ alkoxy, optionally substituted with one to five fluorines, and

C₁₋₄ alkylthio, optionally substituted with one to five fluorines;

R6 is selected from the group consisting of:

—(CH₂)ₘ-aryl,

—(CH₂)ₘ-heteroaryl,

—OCH₂-aryl,

—OCH₂-heteroaryl,

—(S)ᵣCH₂-aryl,

—(S)ᵣCH₂-heteroaryl,

—CH₂O—aryl,

—CH₂O—heteroaryl,

—CH₂(S)ᵣ-aryl, and

—CH₂(S)ᵣ-heteroaryl;

wherein any methylene (CH₂) carbon atom in R6 is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group and wherein aryl and heteroaryl are optionally substituted with one to three Rd substituents independently selected from the group consisting of:

halogen,

cyano,

C₁₋₄ alkyl, optionally substituted with one to five fluorines,

C₁₋₄ alkoxy, optionally substituted with one to five fluorines,

C₁₋₄ alkylthio, optionally substituted with one to five fluorines, and

C₁₋₄ alkylsulfonyl, optionally substituted with one to five fluorines;

each R⁹ is independently selected from the group consisting of hydrogen,

C₁₋₆ alkyl,

(CH₂)ₘ-aryl,

(CH₂)_{m′}-heteroaryl, and

(CH₂)ₘC₃₋₆ cycloalkyl;

wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three substituents independently selected from the group consisting of halogen, C₁₋₄ alkyl, and C₁₋₄ alkoxy; or two R⁹ groups substituents together with the nitrogen atom to which they are attached form a heterocyclic ring selected from azetidine, pyrrolidine, piperidine, piperazine, and morpholine wherein said heterocyclic ring is optionally substituted with one to three substituents independently selected from the group consisting of halogen, hydroxy, C₁₋₆alkyl, and C₁₋₆ alkoxy, wherein alkyl and alkoxy are optionally substituted with one to five fluorines;
each R¹⁰ is independently C₁₋₆ alkyl, wherein alkyl is optionally substituted with one to five substituents independently selected from fluorine and hydroxy;
R¹¹ is hydrogen or R¹⁰;
each n is independently an integer from 0 to 3;
each m is independently an integer from 0 to 2; and
each r is an integer from 0 to 2.

The P2Y14 antagonist may be a triazole derivative, such as one of the compounds described in WO 2017/053769, the contents of which are incorporated herein by reference. Such compounds may be represented by the formula (XI):

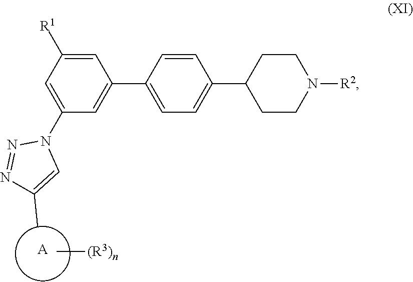

wherein

ring A is aryl, heteroaryl, or cycloalkyl;

R¹ is —CO₂H, —CO₂(C₁-C₈ alkyl), or a bioisostere of carboxylate;

R² is H, C₁-C₈ alkyl, C₂-C₈ alkenyl, C₂-C₈ alkynyl, C₃-C₆ cycloalkyl, C₃-C₆ cycloalkylalkyl, hydroxyalkyl, C₁-C₈ haloalkyl, cyanoalkyl, aryl, heteroaryl, heterocycloalkyl, —(CH₂)ₘaryl, —(CH₂)ₘheteroaryl, or —(CH₂)ₘheterocycloalkyl;

each R³ is the same or different and each is C₁-C₈ alkyl, C₂-C₈ alkenyl, C₃-C₆ cycloalkyl, hydroxy, hydroxyalkyl, C₁-C₈ alkoxy, C₃-C₆ cycloalkyloxy, aryloxy, halo, C₁-C₈ haloalkyl, C₁-C₈ haloalkoxy, —CN, —NO₂, —NR⁵R⁶, —C(O)R⁴, —CO₂R⁴, —C(O)NR⁵R⁶, —NR⁵C(O)R⁴, —(CH₂)ₘaryl, —(CH₂)ₘheteroaryl, or —(CH₂)ₘheterocycloalkyl;

R⁴, R⁵, and R⁶ are the same or different and each is H or C₁-C₈ alkyl; and

m and n are the same or different and each is 0 or an integer from 1-5;

or a pharmaceutically acceptable salt thereof.

Other compounds described in WO 2017/053769 that may be used as P2Y14 antagonists are represented by the formula (XII):

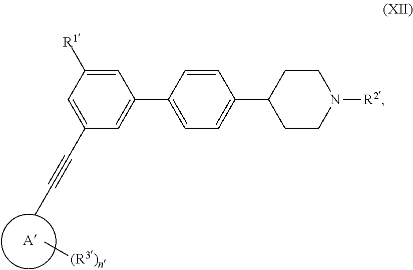

wherein

ring A′ is aryl, heteroaryl, or cycloalkyl;

R¹ is —CO₂H, —O₂(C₁-C₈ alkyl), or a bioisostere of carboxylate;

R¹ is H, C₁-C₈ alkyl, C₂-C₈ alkenyl, C₂-C₈ alkynyl, C₃-C₆ cycloalkyl, C₃-C₆ cycloalkylalkyl, hydroxyalkyl, C₁-C₈ haloalkyl, cyanoalkyl, aryl, heteroaryl, heterocycloalkyl, —(CH₂)_{m′}-aryl, —(CH₂)_{m′}-heteroaryl, or —(CH₂)_{m′}-heterocycloalkyl;

each R^{3′} is the same or different and each is C₁-C₈ alkyl, C₂-C₈ alkenyl, C₃-C₆ cycloalkyl, hydroxy, hydroxyalkyl, C₁-C₈ alkoxy, C₃-C₆ cycloalkyloxy, aryloxy, halo, C₁-C₈ haloalkyl, C₁-C₈ haloalkoxy, —CN, —NO₂, —NR⁵R⁶, —C(O)R^{4′}, —CO₂R^{4′}, —C(O)NR⁵R⁶, —NR⁵C(O)R^{4′}, —(CH₂)_{m′}-aryl, —(CH₂)_{m′}-heteroaryl, or —(CH₂)_{m′}-heterocycloalkyl;

R^{4′}, R^{5′}, and R^{6′} are the same or different and each is H or C₁-C₈ alkyl; and

m′ and n′ are the same or different and each is 0 or an integer from 1-5;

or a pharmaceutically acceptable salt thereof.

The P2Y14 antagonist may be 4-[4-(piperidin-4-yl)phenyl]-7-[4-(trifluoromethyl)phenyl]-2-naphthoic acid (PPTN), a compound having the structure:

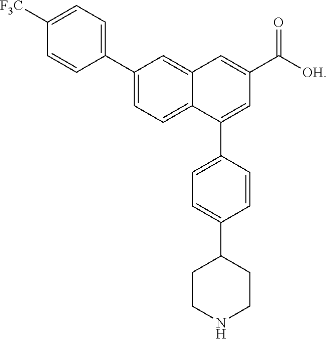 [SMILES: O=C(O)c1cc(-c2ccc(C3CCNCC3)cc2)c2ccc(-c3ccc(C(F)(F)F)cc3)cc2c1]

The P2Y14 antagonist may be a prodrug, analog, derivative, or pharmaceutically acceptable salt of PPTN or of any other active compound that inhibits P2Y14.

Any of the compounds described above may be provided as a pharmaceutically acceptable salt. For example and without limitation, the pharmaceutically acceptable salt may include one or more of acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methanesulfonate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tamiate, tartrate, teoclate, tosylate, triethiodide, and valerate.

The P2Y14 antagonist may be provided in a pharmaceutical composition. A pharmaceutical composition may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions, and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as lactose; granulating and disintegrating agents, for example corn starch, or alginic acid; binding agents, for example starch, gelatin or acacia, and lubricating agents, for example magnesium stearate, stearic acid or talc.

The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. 2003/0232877, which are incorporated by reference herein in their entirety.

Formulations for oral use may also be presented as hard gelatin capsules in which the compounds are mixed with an inert solid diluent, such as kaolin. The formulations may be presented as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The pharmaceutical composition may be formulated for intravenous injection or subcutaneous administration. For example, the compositions may be dissolved, suspended or emulsified. The compositions may also be lyophilized, and the lyophilized material may be used to prepare a formulation for injection. Suitable solvents for injectable formulations include, for example and without limitation, water, physiological saline solution, alcohols, e.g. ethanol, propanol, glycerol, sugar solutions, such as hexose or mannitol solutions, and mixtures of the aforementioned solvents. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic monoglycerides or diglycerides, and fatty acids, including oleic acid.

The pharmaceutical composition may be formulated for delivery of a compound that is insoluble or poorly soluble in water. Examples of such formulations include nanoparticles, microparticles, nanosuspensions, phospholipid-coated microcrystals, emulsions, and stable aqueous formulations. Formulations for delivery of insoluble or poorly soluble compounds are known in the art and described in, for example, U.S. Pat. Nos. 5,091,187; 5,858,410; 8,313,777; 9,308,180; U.S. Publication No. 2002/0012704; U.S. Publication No. 2003/0027858; U.S. Publication No. 2008/0166411; U.S. Publication No. 2010/0093872; U.S. Publication No. 2013/0115165; International Publication No. WO 2014/165660; Pace S. et al., “Novel injectable formulations of insoluble drugs”, Pharm. Tech, 1999, 23:116-134; and Panagiotou T. et al., “Production of stable nanosuspensions using microfluidics reaction technology”, Nanotech. 2007, 4:246-249, ISBN 1420063766, the contents of each of which are incorporated herein by reference.

Pharmaceutical compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The P2Y14 antagonist may be provided as one or more pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

The pharmaceutical composition or formulation may include one or more agents that increase the solubility of a P2Y14 antagonist, such as PPTN, in an aqueous medium. Examples of suitable agents include sulfobutyl ether beta-cyclodextrin (SBECD) or α-tocopherol polyethylene glycol succinate (TPGS).

The presence of the agent may allow the formulation to contain a certain concentration of the P2Y14 antagonist. Thus, the formulation, including the P2Y14 antagonist and the agent, may contain the P2Y14 antagonist at ≥0.001 μg/ml, ≥0.002 μg/ml, ≥0.005 μg/ml, ≥0.01 μg/ml, >0.02 μg/ml, ≥0.05 μg/ml, ≥0.1 μg/ml, ≥0.2 μg/ml, ≥0.5 μg/ml, ≥1 μg/ml, ≥2 μg/ml, ≥5 μg/ml, ≥10 μg/ml, ≥20 μg/ml, ≥50 μg/ml, ≥100 μg/ml, ≥200 μg/ml, ≥500 μg/ml, ≥1 mg/ml, ≥2 mg/ml, ≥5 mg/ml, ≥10 mg/ml, from about 1 μg/ml to about 20 mg/ml, from about 2 μg/ml to about 20 mg/ml, from about 5 μg/ml to about 20 mg/ml, from about 10 μg/ml to about 20 mg/ml, from about 20 μg/ml to about 20 mg/ml, from about 50 μg/ml to about 20 mg/ml, from about 100 μg/ml to about 20 mg/ml, from about 200 μg/ml to about 20 mg/ml, from about 500 μg/ml to about 20 mg/ml, from about 1 mg/ml to about 20 mg/ml, from about 2 mg/ml to about 20 mg/ml, from about 5 mg/ml to about 20 mg/ml, from about 1 μg/ml to about 10 mg/ml, from about 2 μg/ml to about 10 mg/ml, from about 5 μg/ml to about 10 mg/ml, from about 10 μg/ml to about 10 mg/ml, from about 20 μg/ml to about 10 mg/ml, from about 50 μg/ml to about 10 mg/ml, from about 100 μg/ml to about 10 mg/ml, from about 200 μg/ml to about 10 mg/ml, from about 500 μg/ml to about 10 mg/ml, from about 1 mg/ml to about 10 mg/ml, from about 2 mg/ml to about 10 mg/ml, from about 5 mg/ml to about 10 mg/ml, from about 1 μg/ml to about 5 mg/ml, from about 2 μg/ml to about 5 mg/ml, from about 5 μg/ml to about 5 mg/ml, from about 10 μg/ml to about 5 mg/ml, from about 20 μg/ml to about 5 mg/ml, from about 50 μg/ml to about 5 mg/ml, from about 100 μg/ml to about 5 mg/ml, from about 200 μg/ml to about 5 mg/ml, from about 500 μg/ml to about 5 mg/ml, from about 1 mg/ml to about 5 mg/ml, from about 2 mg/ml to about 5 mg/ml, from about 1 μg/ml to about 2 mg/ml, from about 2 μg/ml to about 2 mg/ml, from about 5 μg/ml to about 2 mg/ml, from about 10 μg/ml to about 2 mg/ml, from about 20 μg/ml to about 2 mg/ml, from about 50 μg/ml to about 2 mg/ml, from about 100 μg/ml to about 2 mg/ml, from about 200 μg/ml to about 2 mg/ml, from about 500 μg/ml to about 2 mg/ml, or from about 1 mg/ml to about 2 mg/ml.

The agent may promote solubility of the P2Y14 antagonist at a near-neutral pH. Thus, the formulation, including the P2Y14 antagonist and the agent, may have a pH of ≥4.0, ≥4.5, >5.0, ≥5.5, ≥6.0, ≥6.5, ≥7.0, ≥7.5, ≥8.0, from about 4.0 to about 9.0, from about 5.0 to about 9.0, from about 6.0 to about 9.0, from about 7.0 to about 9.0, from about 4.0 to about 8.0, from about 5.0 to about 8.0, from about 6.0 to about 8.0, from about 7.0 to about 8.0, from about 4.0 to about 7.0, from about 5.0 to about 7.0, from about 6.0 to about 7.0, about 5.0, about 5.5, about 6.0, about 6.5, about 7.0, about 7.5, or about 8.0.

The agent may be present in the formulation at a certain concentration. For example, the agent may be present in the formulation at less than about 40%, less than about 35%, less than about 30%, less than about 25%, less than about 20%, less than about 15%, less than about 10%, less than about 5%, less than about 2%, less than about 1%, less than about 0.5%, less than about 0.2%, less than about 0.1%, less than about 0.05%, less than about 0.02%, less than about 0.01%, less than about 0.005%, less than about 0.002%, less than about 0.001%, from about 0.001% to about 0.01%, from about 0.003% to about 0.03%, from about 0.01% to about 0.1%, from about 0.03% to about 0.3%, from about 0.1% to about 1%, from about 0.3% to about 3%, from about 1% to about 10%, from about 2% to about 10%, from about 3% to about 10%, from about 5% to about 10%, from about 5% to about 12%, from about 5% to about 15%, from about 5% to about 20%, from about 7.5% to about 10%, from about 7.5% to about 12%, from about 7.5% to about 15%, from about 7.5% to about 20%, from about 10% to about 12%, from about 10% to about 15%, or from about 10% to about 20%.

The agent may improve the stability of the P2Y14 antagonist. For example, the agent may increase the half-life of the P2Y14 antagonist by about 10%, about 25%, about 50%, about 100%, about 200%, about 500%, about 1000%, or more.

The formulation may contain dimethyl sulfoxide (DMSO). The formulation may contain DMSO at or below a certain concentration. For example, DMSO may be present in the formulation at less than about 10%, less than about 5%, less than about 3%, less than about 2%, less than about 1%, less than about 0.5%, less than about 0.3%, less than about 0.2%, or less than about 0.1%.

The formulation may be substantially free of solvents or other chemicals that are not suitable for administration to a subject. For example, the formulation may be substantially free of dimethylacetamide (DMAc), ethanol, N-methylpyrrolidone (NMP), and/or polyethylene glycol (PEG).

Formulations that contain P2Y14 antagonists are described in, for example, co-owned, co-pending U.S. Patent Application No. 62/834,517, the contents of which are incorporated herein in their entirety.

Renal Inflammation Associated with Acute Kidney Injury (AKI)

The methods may treat or prevent renal inflammation associated with AKI. AKI may be assessed by any suitable standard. Several standards for acute kidney injury are known in the art, such as the criteria provided by the Acute Kidney Injury Network (AKIN); Kidney Disease Improving Global Outcomes (KDIGO); and Risk, Injury, Failure, Loss, and End-stage Kidney (RIFLE). AKI may be categorized or staged according to the AKI, KDIGO, or RIFLE criteria. For example, a subject may be deemed to have stage 1, stage 2, or stage 3 AKI, or a subject may be deemed to have risk, injury, failure, or loss. The standard may apply to an adult, pediatric, newborn, neonatal, infant, child, adolescent, pre-teen, teenage, or elderly subject.

Standards typically include measurements of serum creatinine (SCr) concentrations, urine output, or glomerular filtration rate (GFR). Standards may include multiple parameters, e.g., combinations of the aforementioned standards. A subject may be deemed to have AKI, or a stage or category thereof, when she has abnormally high SCr concentration, abnormally low urine output, abnormally low GFR, or any combination thereof. Standards may be absolute, e.g., they may require a value above or below a defined threshold value. Alternatively, standards may be relative, e.g., they may require an increase or decrease relative to a baseline value. Standards for different parameters, e.g., abnormally high SCr concentration abnormally low urine output, or abnormally low GFR, may independently be absolute or relative.

Standards for acute kidney injury may include a temporal component. For example, a subject may be deemed to have AKI when an elevated SCr concentration is measured at some interval following a preceding event. The preceding event may be cardiac surgery, cardiac arrest, admission to a hospital, clinic, medical facility, or any unit thereof. The interval may be 1 hour, 2 hours, 4 hours, 6 hours, 8 hours, 12 hours, 24 hours, 36 hours, 48 hours, or 72 hours. A subject may be deemed to have AKI when urine output is measured across some interval, such as 1 hour, 2 hours, 4 hours, 6 hours, 8 hours, 12 hours, 24 hours, 36 hours, 48 hours, or 72 hours.

For example and without limitation, a standard for reduced urine output associated with AKI may be less than 0.5 mL/kg/h for 6-12 hours, less than 0.5 mL/kg/h for at least 12 hours, or less than 0.3 mL/kg/h for 24 hours, or anuria for at least 12 hours.

For example and without limitation, a standard for elevated SCr concentration associated with AKI may be a SCr concentration of at least 0.3 mg/dl, a SCr concentration of at least 1 mg/dl, a SCr concentration of at least 4 mg/dl, a SCr concentration of at least 26.5 μmol/l, or a SCr concentration of at least 353.6 μmol/l. For example and without limitation, a standard for elevated SCr concentration associated with AKI may be an increase of 50% over baseline, an increase of 100% over baseline, or an increase of 200% over baseline.

For example and without limitation, a standard for GFR associated with AKI may be a GFR of less than 35 ml/min per 1.73 mm². For example and without limitation, a standard for GFR associated with AKI may be a decrease of at least at least 25% relative to a baseline, a decrease of at least at least 50% relative to a baseline, or a decrease of at least at least 75% relative to a baseline.

### Improvement of Renal Function

Providing a P2Y14 antagonist may improve renal function. For example, renal function may be improved by at least 10%, at least 20%, at least 30%, at least 40%, at least 50%, at least 60%, at least 70%, at least 80%, at least 90%, at least 100%, at least 200%, or at least 300%. Measurable markers of renal function, are well known in the medical and veterinary literature and to those of skill in the art, and include, but are not limited to, blood urea nitrogen or “BUN” levels (both static measurements and measurements of rates of increase or decrease in BUN levels), serum creatinine levels (both static measurements and measurements of rates of increase or decrease in serum creatinine levels), measurements of the BUN/creatinine ratio (static measurements of measurements of the rate of change of the BUN/creatinine ratio), urine/plasma ratios for creatinine, urine/plasma ratios for urea, glomerular filtration rates (GFR), serum concentrations of sodium (Na⁺), urine osmolarity, daily urine output, albuminuria, proteinuria, and the like. Of the above, measurements of the plasma concentrations of creatinine and/or urea or BUN are particularly important and useful readouts of renal function.

### Treating and Preventing Reperfusion Injury

Reperfusion injury, which is also called reperfusion insult, ischemia-reperfusion injury, and reoxygenation injury, is the tissue damage that results when blood supply to the tissue is restored after a period of ischemia or lack of oxygen. The sudden influx of nutrients and oxygen after a bout of ischemia, anoxia, or hypoxia produces a high level of reactive oxygen species that exceeds the tissue's detoxification capacity. The oxidative stress is associated with microvascular injury due to increased permeability of capillaries and arterioles that allows fluid to penetrate the tissue more readily. In addition, white blood cells in the returning blood respond to damaged tissue by releasing inflammatory factors.

Reperfusion injury can occur following any surgery that limits blood supply to an organ. In particular, reperfusion injury is a risk following cardiac procedures due to changes in blood during the procedure. Reperfusion injury is also a major concern in organ transplantation procedures due to the lack of blood flow to the organ while it is being transported.

Certain organs are particularly vulnerable to reperfusion injury. For example, reperfusion injury contributes to the brain's ischemic cascade in stroke or brain trauma, and also plays a role in brain damage following cardiac arrest. The heart, kidneys, lungs, and liver may also be affected by reperfusion injury.

Without wishing to be bound by a particular theory, P2Y14 antagonists may prevent or minimize reperfusion injury by preserving microvascular integrity. Therefore, it may be advantageous to provide a P2Y14 antagonist, such as any of the P2Y14 antagonists described above, to a patient before the patient undergoes a surgery that is likely to cause reperfusion injury. The surgery may be a cardiac procedure, such as any of the cardiac procedures described above. The P2Y14 antagonist may be provided by any mechanism described above.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for monitoring for development of renal inflammation in a subject that has undergone a cardiac procedure or has had a cardiac event, the method comprising:
obtaining a sample from a subject that has undergone a cardiac procedure or has had a cardiac event;
conducting an assay on the sample to measure a level of a UDP-hexose in the sample; and
comparing the level of the UDP-hexose from the sample with a reference level of a UDP-hexose, wherein an elevated level of the UDP-hexose indicates that the subject is at risk of developing or has developed renal inflammation.

2. The method of claim 1, wherein the subject is a critical care subject.

3. The method of claim 1, wherein the cardiac procedure is selected from the group consisting of an aneurysm repair, angioplasty, cardiomyoplasty, coronary artery bypass graft, heart transplant, heart valve repair, heart valve replacement, implantation of a defibrillator, implantation of a total artificial heart, implantation of a ventricular assist device, implantation of pacemaker, and transmyocardial laser revascularization.

4. The method of claim 1, wherein the cardiac event is a myocardial infarction.

5. The method of claim 1, further comprising repeating the obtaining, conducting, and comparing steps to thereby monitor the subject over time.

6. The method of claim 1, wherein the reference level is an average of the UDP-hexose level in a population of healthy subjects.

7. The method of claim 1, wherein the reference level is the subject's own UDP-hexose level prior to undergoing the cardiac procedure.

8. The method of claim 1, wherein the sample is a body fluid sample.

9. The method of claim 1, further comprising providing a P2Y14 receptor antagonist to the subject if the subject has an elevated level of UDP-hexose.

10. The method of claim 9, wherein the P2Y14 antagonist is 4-((piperidin-4-yl)-phenyl)-(7-(4-(trifluoromethyl)-phenyl)-2-naphthoic acid (PPTN).

11. The method of claim 1, wherein the UDP-hexose is at least one compound selected from the group consisting of UDP-glucose, UDP-galactose, UDP-glucuronic acid, N-acetyl-UDP-glucosamine, N-acetyl-UDP-galactosamine, and combinations thereof.

12. A method of treating or preventing renal inflammation in a subject who has undergone or will undergo a cardiac procedure or has experienced a cardiac event, the method comprising:
providing a P2Y14 antagonist to a subject who has undergone or will undergo a cardiac procedure or has experienced a cardiac event.

13. The method of claim 12, wherein the P2Y14 antagonist is a substituted 2-naphthoic acid.

14. The method of claim 13, wherein the P2Y14 antagonist is 4-((piperidin-4-yl)-phenyl)-(7-(4-(trifluoromethyl)-phenyl)-2-naphthoic acid (PPTN).

15. The method of claim 12, wherein the cardiac procedure is selected from the group consisting of an aneurysm repair, angioplasty, cardiomyoplasty, coronary artery bypass graft, heart transplant, mitral valve replacement, heart valve repair, heart valve replacement, implantation of a defibrillator, implantation of a total artificial heart, implantation of a ventricular assist device, implantation of pacemaker, and transmyocardial laser revascularization.

16. The method of claim 12, wherein the cardiac event is a myocardial infarction

17. The method of claim 12, wherein the renal inflammation is associated with acute kidney injury.

18. The method of claim 12, wherein the cardiac procedure or cardiac event is associated with elevated levels of one or more UDP-hexose in the subject.

19. The method of claim 12, further comprising: measuring a level of a UDP-hexose in a body fluid sample from the subject.

20. The method of claim 19, wherein the UDP-hexose is at least one compound selected from the group consisting of UDP-glucose, UDP-galactose, UDP-glucuronic acid, N-acetyl-UDP-glucosamine, N-acetyl-UDP-galactosamine, and combinations thereof.

21. The method of claim 19, wherein the body fluid is blood or urine.

22. The method of claim 21, wherein the body fluid is urine.

23-47. (canceled)
